(19) United States
(12) Patent Application Publication
(10) Pub. No.: US 2005/0277823 A1
(43) Pub. Date: Dec. 15, 2005

(54) Angiogram display overlay technique for tracking vascular intervention sites

(76) Inventors: Robert Sutherland, Menlo Park, CA (US); David Hall, Menlo Park, CA (US); Hassan Mostafavi, Los Altos, CA (US)

Correspondence Address: Daniel E. Ovanezian;BLAKELY, SOKOLOFF, TAYLOR & ZAFMAN LLP, Seventh Floor, 12400 Wilshire Boulevard, Los Angeles, CA, 90025-1026, US

(21) Appl. No.: 10/391,171

(22) Filed: Mar. 17, 2003

Related U.S. Application Data
(60) Provisional application No. 60/387,663, filed on Jun. 10, 2002.

(51) Int. Cl. A61B 5/05

(52) U.S. Cl. 600/407

(57) ABSTRACT

A method and apparatus for tracking vascular intervention sites is described. A vascular site is selected and marked on a first image of an angiogram display. The vascular site may be identified on a second image of the angiogram display.

### REFERENCE TO RELATED APPLICATION

This application claims the benefit of U.S. Provisional Application No. 60/387,663, filed Jun. 10, 2002.

### FIELD OF THE INVENTION

This invention relates to the field of angiography and, in particular, to tracking vascular intervention sites.

### BACKGROUND OF THE INVENTION

Coronary artery disease involves narrowing in an artery that causes a decrease in the flow of blood to the heart. Diagnostic methods such as angiography may be employed if coronary artery disease is suspected. In angiography, a dye is injected into a patient's coronary arteries through a catheter or flexible tube that is inserted into a main artery and guided to the heart. A user can then use an x-ray or angiogram to discover any narrowing in the arteries by analyzing how the dye traveled through the vessel.

In treating advanced cases of coronary artery disease, measures such as balloon angioplasty may be employed. Balloon angioplasty is a procedure used by cardiologists to open blocked arteries in the heart, as illustrated in FIG. 1. Artery 100 is healthy and displays no sign of narrowing. Artery 110 has a partial blockage. In balloon angioplasty, a small balloon 125 is passed through a catheter into the blocked area of an artery 120 in order to compress the plaque against the artery wall, thereby stretching the blockage open, as in artery 130. One problem with balloon angioplasty is the significant chance that the blockage could return, even after a perfect initial result, within the first six months after dilation. This is due to the natural healing process of the artery. Should a blockage recur, balloon angioplasty can be repeated or coronary stenting can be performed. This would, however, require additional interventions.

An intracoronary stent, as illustrated in arteries 140 and 150, is a small wire “scaffolding” that is mounted on a small balloon catheter. The balloon is used to deliver the stent to the desired location inside a coronary artery. Once the stent has been delivered to the desired site the balloon is inflated, thereby expanding the stent and embedding it into the wall of the artery. The balloon is then deflated and removed, leaving the stent permanently implanted.

Restenosis, or the repeated blockage of blood vessels after balloon angioplasty or coronary stenting, is one of the greatest challenges of interventional cardiology and radiology. Artery 150 has a re-narrowed passageway 160, even though the artery has a stent 155 in place. One means of reducing the restenosis rate in a patient and, thereby, help to avoid repeated interventions, is to apply endovascular brachytherapy (EVBT) after balloon angioplasty or stent placement. EVBT is illustrated in FIG. 2. EVBT is a form of radiotherapy whereby a radioactive source, usually in the form of radioactive seeds 250, is positioned within a treatment area 230 having blockage 240 for a predetermined amount of time. Although EVBT may reduce the restenosis rate, some patients continue to experience restenosis even after EVBT.

Restenosis at the treatment margin, or edge effect, is particularly significant. Edge effect, or candy-wrapper effect, is the failure of radiotherapy to prevent restenosis at the edges of a lesion. Edge effect is illustrated in FIG. 3. A blood vessel 300, having previously received angioplasty treatment and possibly subsequent EVBT, is now experiencing restenosis at the proximal and distal edges of the original injured area, as represented by growths 310 and 320. Without proper EVBT over the entire injured area, either of the two growths could become a complete blockage.

One hypothesis for edge effect is inadequate dose during treatment. Factors contributing to the underdose may include longitudinal seed movement and barotrauma. Longitudinal seed movement refers to movement of the radioactive seeds relative to the coronary vessel during the cardiac cycle. During EVBT, the delivery catheter is anchored on the patient's thigh and floats freely inside the coronary vessel. As the heart contracts and expands during each cardiac cycle, the delivery catheter and radioactive seeds may move with the blood flow, which may result in inadequate dose. Barotrauma refers to injury to the vessel arising from interventions such as balloon angioplasty or stent placement. The length of the balloon used for stent deployment is typically longer than the stent itself. This can lead to injury to the vessel extending beyond the injury inflicted from the stent. Other interventions, such as use of a Roto-Blator or even vigorous interference from a guide wire or catheter delivery, can also cause blood vessel damage. Barotrauma may be a contributing factor to inadequate dose during EVBT.

Currently, cardiologists, radiologists, or other clinicians apply vascular brachytherapy to intervention sites based on “injured length,” which is defined as the length of a stent, stenotic lesion, or barotrauma. FIG. 4 illustrates a blood vessel 400 having an injured length 410 corresponding to placement of a stent 455, a proximal margin 460, and a distal margin 470. Presently, the proximal and distal margins, which are not necessarily the same, are not tracked. Therefore, the full extent of the injury may not receive the necessary radiotherapy. Such incomplete treatment may lead to edge effect, which would eventually require additional radiotherapy and, therefore, additional interventions. A desired treatment length 420 would include the injured length and the proximal and distal margin lengths to ensure a maximally effective treatment by radioactive seeds 480.

Another problem currently faced is that, because proximal and distal margins are not tracked, subsequent treatments may fail to take edge effect into account. There is a difficulty in determining where these intervention damage sites might lie, but it appears necessary that radiation be administered to all damaged sites. If the proximal and distal margins are not sufficiently tracked, as is the case presently, complete and adequate radiotherapy is very difficult.

Over time, determining the extent of intervention damage sites becomes even more difficult. This is because, without an effective way to track the full extent of an intervention damage site, determination of the extent of the damage site in each angiogram is a subjective judging based heavily on observation alone. Such determination may be erroneous due to changes in imaging geometry and movement of coronary vessels, for examples, which are very difficult to observe. Such determinations may also be inconsistent due to, for examples, different doctors and changes in observation standards. Improper or incomplete determinations of an intervention damage site may result in inadequate treatment that may lead to edge effect, which would eventually require additional radiotherapy and, therefore, additional interventions.

### SUMMARY OF THE INVENTION

The present invention pertains to a method and apparatus for tracking vascular intervention sites. In one embodiment, the method may include selecting a vascular site and marking the vascular site on a first image of an angiogram display. The vascular site may be identified on a second image of the angiogram display.

Additional features and advantages of the present invention will be apparent from the accompanying drawings, and from the detailed description that follows below.

### BRIEF DESCRIPTION OF THE DRAWINGS

The present invention is illustrated by way of example, and not by way of limitation, in the figures of the accompanying drawings.

FIG. 1 illustrates balloon angioplasty administered to a vascular intervention site.

FIG. 2 illustrates endovascular brachytherapy administered to a vascular intervention site.

FIG. 3 illustrates edge, or candy-wrapper, effect.

FIG. 4 illustrates a treatment source length.

FIG. 5 illustrates one embodiment of an apparatus used in tracking vascular intervention sites.

FIG. 6A illustrates one embodiment of virtual markers placed on an angiogram display to track vascular intervention sites.

FIG. 6B illustrates one embodiment of a single angiogram display used to display two viewed positions of an anatomical landmark through use of the virtual markers of FIG. 6A.

FIG. 6C illustrates one embodiment of two angiogram displays used to display two viewed positions of an anatomical landmark through use of the virtual markers of FIG. 6A.

FIGS. 7A-7C illustrate a display generated by one embodiment of algorithms and software for tracking the pattern of an anatomical landmark including a vascular treatment site from one image to another.

FIG. 7A illustrates an example of a reference trace and a second viewing trace on the same x,y coordinates.

FIG. 7B illustrates a 1-D function generated from the reference trace points surrounding the designated landmark of FIG. 7A.

FIG. 7C illustrates the 1-D function of FIG. 7B for the second viewing trace.

FIG. 8A illustrates one embodiment of a blood vessel having a first position.

FIG. 8B illustrates one embodiment of the blood vessel of FIG. 8A having a second position.

FIG. 9A illustrates one embodiment of a first position of a blood vessel having a stent within a vascular intervention area.

FIG. 9B illustrates one embodiment of a second position of the blood vessel of FIG. 9A.

FIG. 10 illustrates one embodiment of a digital processing system used in tracking vascular intervention sites.

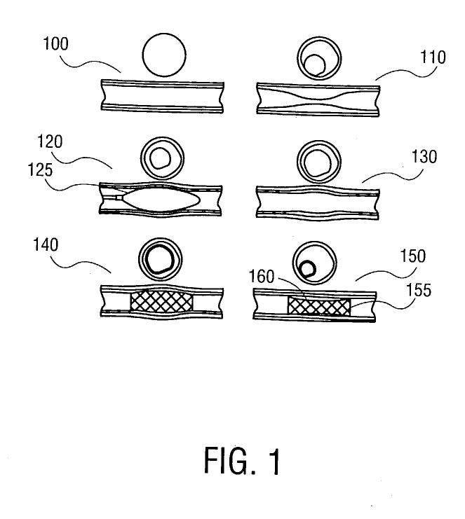

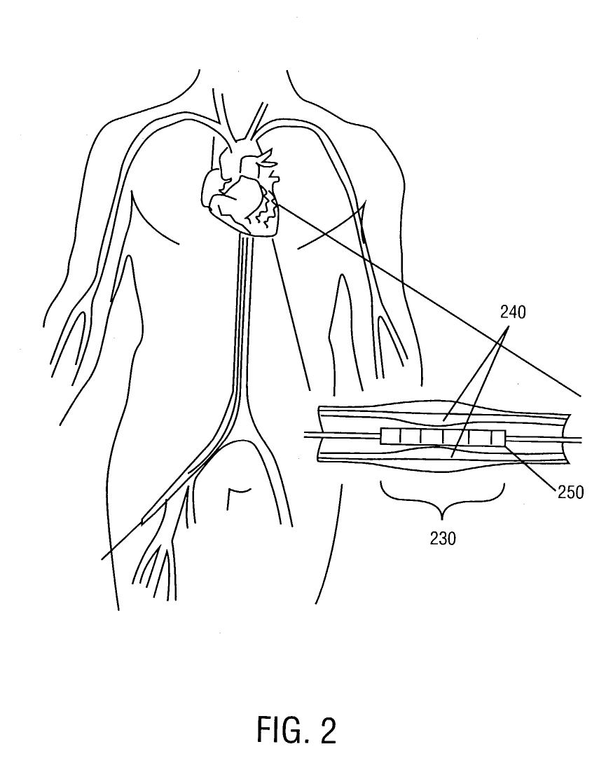

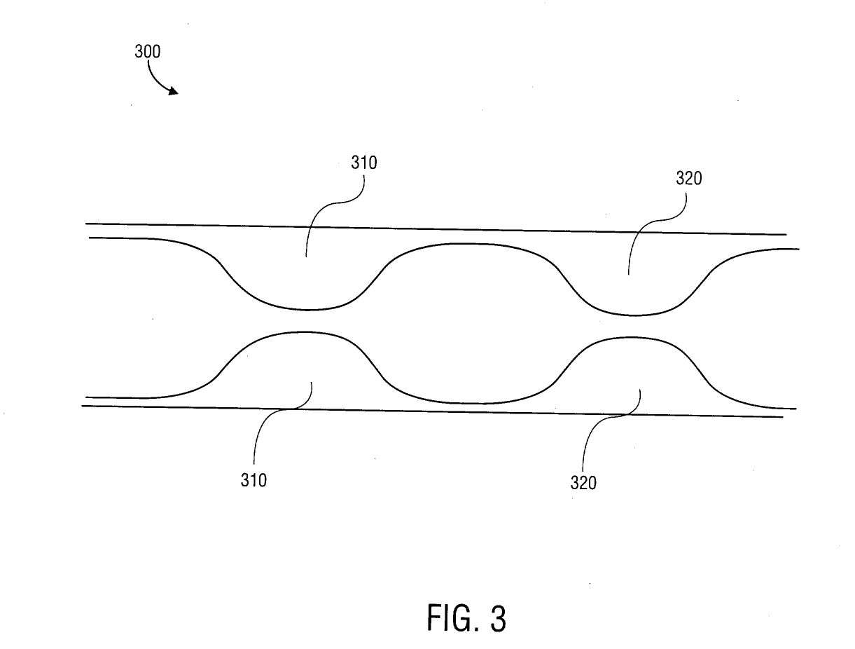

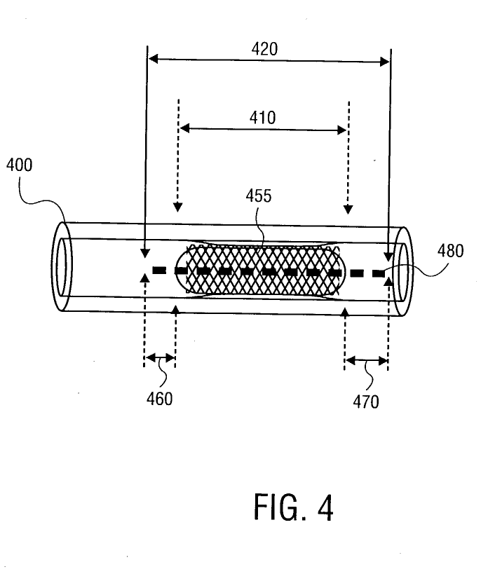

### DETAILED DESCRIPTION

In the following description, numerous specific details are set forth such as examples of specific systems, components, methods, etc. in order to provide a thorough understanding of the present invention. It will be apparent, however, to one skilled in the art that these specific details need not be employed to practice the present invention. In other instances, well-known components or methods have not been described in detail in order to avoid unnecessarily obscuring the present invention.

The present invention includes various steps, which will be described below. The steps of the present invention may be performed by hardware components or may be embodied in machine-executable instructions, which may be used to cause a general-purpose or special-purpose processor programmed with the instructions to perform the steps. Alternatively, the steps may be performed by a combination of hardware and software.

The present invention may be provided as a computer program product, or software, that may include a machine-readable medium having stored thereon instructions, which may be used to program a computer system (or other electronic devices) to perform a process according to this present invention. A machine-readable medium includes any mechanism for storing or transmitting information in a form (e.g., software, processing application) readable by a machine (e.g., a computer). The machine-readable medium may include, but is not limited to, magnetic storage medium (e.g., floppy diskette); optical storage medium (e.g., CD-ROM); magneto-optical storage medium; read-only memory (ROM); random-access memory (RAM); erasable programmable memory (e.g., EPROM and EEPROM); flash memory; electrical, optical, acoustical, or other form of propagated signal (e.g., carrier waves, infrared signals, digital signals, etc.); or other type of medium suitable for storing electronic instructions.

The present invention may also be practiced in distributed computing environments where the machine-readable medium is stored on and/or executed by more than one computer system. In addition, the information transferred between computer systems may either be pulled or pushed across the communication medium connecting the computer systems, such as in a remote diagnosis or monitoring system. In remote diagnosis or monitoring, a user may utilize the present invention to diagnose or monitor a patient despite the existence of a physical separation between the user and the patient.

Some portions of the description that follow are presented in terms of algorithms and symbolic representations of operations on data bits that may be stored within a memory and operated on by a processor. These algorithmic descriptions and representations are the means used by those skilled in the art to effectively convey their work. An algorithm is generally conceived to be a self-consistent sequence of acts leading to a desired result. The acts are those requiring manipulation of quantities. Usually, though not necessarily, these quantities take the form of electrical or magnetic signals capable of being stored, transferred, combined, compared, and otherwise manipulated. It has been proven convenient at times, principally for reasons of common usage, to refer to these signals as bits, values, elements, symbols, characters, terms, numbers, parameters, or the like.

A method and apparatus for tracking vascular intervention sites is described. In one embodiment, the method and apparatus is described as a system allowing a user, such as a cardiologist, radiologist, or other clinician, to mark an anatomical landmark containing a vascular intervention site on an angiogram display. A first pattern of the anatomical landmark including the vascular treatment site is determined. Data pertaining to the first pattern may be stored and later recalled. During a subsequent angiogram, a second pattern of the anatomical landmark may be determined and then matched with the first pattern to identify the vascular treatment site. The vascular treatment site may be identified through the use of virtual markers on the angiogram display.

In one embodiment, the virtual markers are automatically displayed on the angiogram display. The user may use the virtual markers on the display as a reference for applying radiotherapy. The ability to monitor the exact location of a vascular treatment site allows the user to administer the proper extent of radiotherapy to prevent against edge effect. In an alternative embodiment, the user may adjust the location and extent of the vascular treatment site by adjusting the virtual markers on the angiogram display after the automatic marking but before applying radiotherapy.

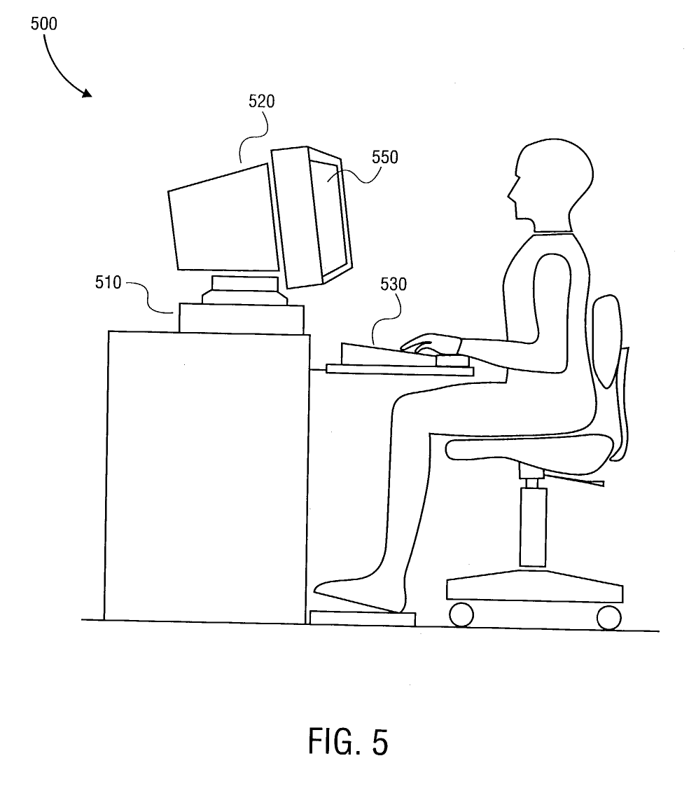

FIG. 5 illustrates one embodiment of an apparatus 500 used in tracking vascular intervention sites. Apparatus 500 comprises a digital processing system 510, a display unit 520, and an input device 530. In one embodiment, digital processing system 510 may be a personal computer, display unit 520 may be a computer monitor, and input device 530 may be a keyboard. Input device 530 may also include other peripheral devices such as a mouse or a light pen. The display unit 520 includes a display screen 550. Display screen 550 may provide an output display of an angiogram. The output display may include virtual markers, such as actual lines or areas of different colors or different shades of colors.

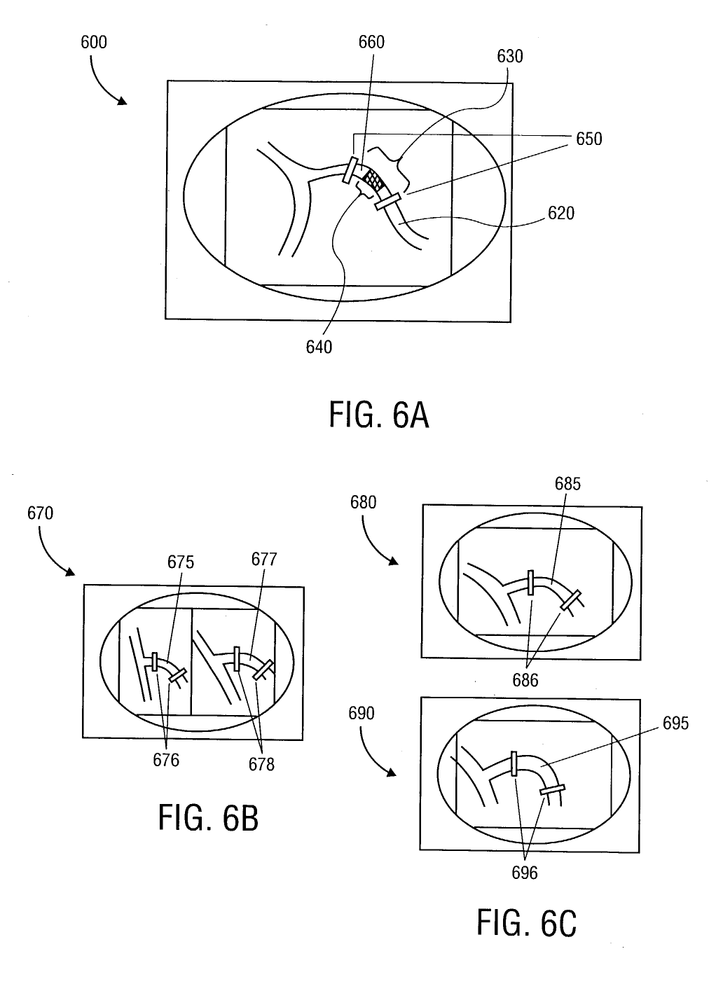

A user may use display screen 550 to view and analyze an angiogram. A user may further use display screen 550 to select an anatomical landmark. In one embodiment, the anatomical landmark may be a segment of a blood vessel that, for example, contains a site to be treated with cardiovascular brachytherapy. The user may select the anatomical landmark by marking it on display screen 550 using input device 530, as discussed below in relation to FIG. 6A. After a subsequent angiogram is taken, digital processing system 510 may recall and process data to identify the anatomical landmark. Apparatus 500 may use virtual markers on display screen 550 to identify the anatomical landmark and thus the treatment site.

FIG. 6A illustrates one embodiment of virtual markers 650 placed on an angiogram display 600 to track vascular intervention sites. The angiogram display 600 may be the output of a display unit such as display unit 520 of FIG. 5. The angiogram display may show at least one blood vessel 620 including at least one anatomical landmark 630. The anatomical landmark 630 contains at least a vascular intervention site 640 and may contain at least one anatomical feature. For example, anatomical landmark 630 may contain a bend 660 in the blood vessel 620. In alternative embodiments, anatomical landmark 630 may contain different and/or additional anatomical features (e.g., a vessel branch). A user may select the anatomical landmark 630 on angiogram display 600 by marking the anatomical landmark 630. This may be performed by the placement of virtual markers 650 on the angiogram display 600.

The virtual markers 650 may be placed in one of several ways. In one embodiment, for example, the virtual markers 650 are placed on the angiogram display 600 through manual marking performed by use of an input device, such as a mouse or light pen, as represented by input device 530 of FIG. 5. There are several ways a user may mark anatomical landmark 630 with an input device. For example, a user may click and drag a cursor from one end of anatomical landmark 630 to the other end. Another technique may be to click once at one end of anatomical landmark 630 and once at the other end.

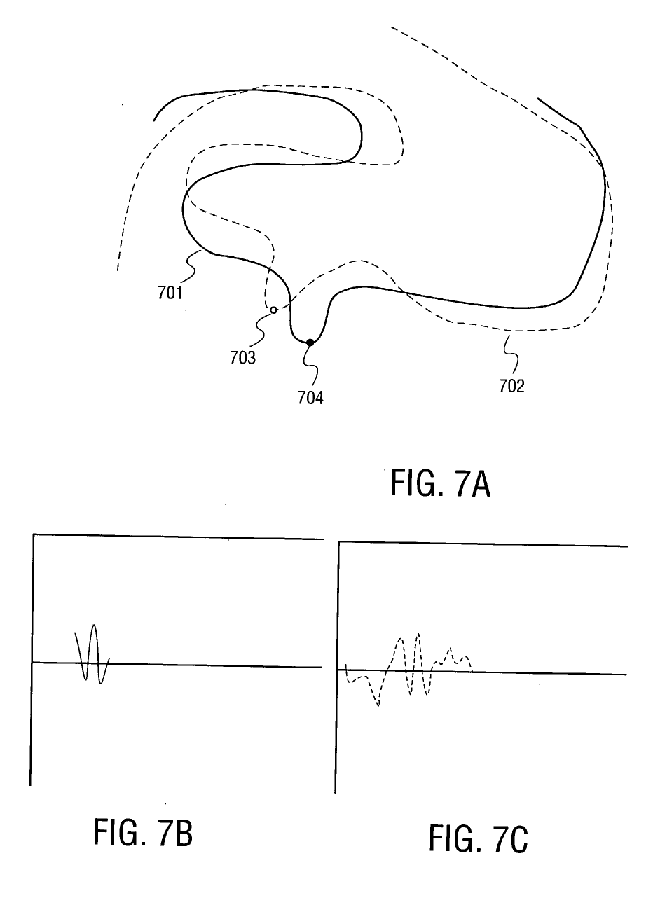

Data defining the virtual markers 650 may be stored for later use. At a later time, for example a subsequent intervention, a user may want to retrieve the data and thus have virtual markers displayed again on an angiogram display. The angiogram display may or may not be the same unit as previously used. Execution of the steps discussed below, in relation to FIGS. 7A-7C, may be performed such that a pattern of an anatomical landmark containing a vascular treatment site may be determined. The pattern may be matched with a previous pattern of the anatomical landmark to identify the vascular intervention site. The location of a vascular intervention site may then be identified on an angiogram display by way of virtual markers.

FIG. 6B illustrates one embodiment of a single angiogram display used to display two viewed positions of an anatomical landmark through use of virtual markers. Angiogram display 670 may be a split-screen display that displays a first viewed position of an anatomical landmark 675 by way of virtual markers 676 on one side and a second viewed position of the anatomical landmark 677 by way of virtual markers 678 on the other side. Alternatively, other means may be used to display viewed positions of an anatomical landmark, for example, as discussed below in relation to FIG. 6C.

FIG. 6C illustrates one embodiment of two angiogram displays used to display two viewed positions of an anatomical landmark through use of virtual markers. A first angiogram display 680 may display a first viewed position of an anatomical landmark 685 by way of virtual markers 686. A second angiogram display 690 may display a second viewed position of the anatomical landmark 695 by way of virtual markers 696. There are several ways in which a user may employ two angiogram displays. For example, the displays may be placed side by side during administration of radiotherapy. In another embodiment, the displays may be kept separate from each other. For example, a first display may be behind the person applying radiotherapy so that the person can look at a previous position of the treatment area for reference, while a second display may be in front of the person applying radiotherapy so that the person can see the area to be treated.

FIGS. 7A-7C illustrate a display generated by one embodiment of software algorithms and the intermediate one-dimensional (1-D) functions for matching a pattern of an anatomical landmark including a vascular treatment site. The calculations described below are used to match at least two patterns of an anatomical landmark to identify a vascular treatment site.

In one embodiment, for example, the user may designate a landmark on a blood vessel in the first viewing such as by a mouse click on the image. The software may then automatically segment the blood vessel in, for example, both directions from the mouse click point and create a trace, or sequence of points, designating the centerline of the vessel. This can be done using one of several automated or semi-automated segmentation techniques that are known to one of ordinary skilled in the art. Alternatively, this could be done simply by manual tracing of the blood vessel. In one embodiment, the length of the trace should be sufficiently large to include a minimum of two local inflection points plus some margin as prescribed by the processing algorithm described below.

$$Let$$

$$\{ ( {X}_{n}^{R} , {Y}_{n}^{R} ) , n = 0 , 1 , … \}$$

denote the reference trace created from the first viewing. In creating this trace, the algorithm ensures that the distance between subsequent points is a constant, i.e.,

$${[ {( {X}_{n + 1}^{R} - {X}_{n}^{R} )}^{2} + {( {Y}_{n + 1}^{R} - {Y}_{n}^{R} )}^{2} ]}^{1 / 2} = Δ , fixed$$

To find the landmark designated in the first viewing in a subsequent image, again a trace is created from portion of the blood vessel that is expected to contain the reference landmark. This second trace is denoted by:

$$\{ ( {X}_{n}^{S} , {Y}_{n}^{S} ) , n = 0 , 1 , … \}$$

The landmark designated on the reference trace is identified by an index, n_{R}, in the reference sequence. The automatic landmark detection algorithm finds the corresponding index, ns, in the trace obtained from the second viewing.

A one-dimensional function may be extracted from each trace to obtain the index. In one embodiment, using cross correlation matching of the two functions, the position of the reference landmark in the second trace is located.

The function that extracts the one-dimensional sequence,
{rₙ, n=0,1, . . . }
from the reference trace is defined as:

$${r}_{n} = {[ {( {X}_{n}^{R} - {\overline{X}}_{n}^{R} )}^{2} + {( {Y}_{n}^{R} - {\overline{Y}}_{n}^{R} )}^{2} ]}^{1 / 2} \cdot Sign ( {A}_{n} {X}_{n - 1 ,}^{R} {B}_{n} {Y}_{n - 1}^{R} + {C}_{n} )$$

$$Where$$

$${\overline{X}}_{n}^{R} = \frac{{\sum }_{m = n - w}^{W} {X}_{m}^{R}}{2 w + 1} , {\overline{Y}}_{n}^{R} = \frac{{\sum }_{m = n - w}^{W} {y}_{m}^{R}}{2 w + 1}$$

$${A}_{n} = ( {\overline{Y}}_{n}^{R} - {Y}_{n}^{R} )$$

$${B}_{n} = - ( {\overline{X}}_{n}^{R} - {X}_{n}^{R} )$$

$${C}_{n} = {\overline{Y}}_{n}^{R} ( {\overline{X}}_{n}^{R} - {X}_{n} ) - {\overline{X}}_{n}^{R} ( {\overline{Y}}_{n}^{R} - {Y}_{n}^{R} )$$

This function in effect computes the distance between a point and the centroid of a 2w+1 long segment of the sequence centered on that point, i.e. neighboring points. The Sign multiplier causes the sequence to be bipolar and have both positive and negative sign depending on whether the centroid is on the right or left of the ray extending from point

$$( {X}_{n - 1}^{R} , {Y}_{n - 1}^{R} ) to ( {X}_{n}^{R} , {Y}_{n}^{R} ) .$$

FIG. 7A illustrates an example of a reference trace 701 and a second viewing trace 702 on the same x,y coordinates. The landmark is shown as a small circle 703 on the reference trace 701. The small circle 704 on the second viewing trace 702 shows the corresponding location as found by the algorithm.

FIG. 7B illustrates a one-dimensional function generated from the reference trace points surrounding the designated landmark shown in FIG. 7A. In one embodiment, the sequence should be long enough to include variations, preferably unique variations, needed for successful matching using cross correlation. This length plus the length of the window used for centroid computation in the equation for \overline{X}ₙ^{R} and \overline{Y}ₙ ^{R} defines the minimum blood vessel length that must be segmented in the reference, or first viewing, image.

Using the same function as defined above, the 1-D sequence, {Sₙ, n=0,1, . . . }, is generated from trace 702 in the second viewing. FIG. 7C illustrates the 1-D function for the second viewing trace 702 of FIG. 7A. To use cross correlation for a search of the best match, this sequence should be longer than the reference 1-D sequence.

The matching algorithm computes the inner product of the reference 1-D sequence and the 1-D sequence from the second viewing at different offsets, and searches for the offset that yields the maximum value of the inner product:

$${m}_{R} = {\sum }_{n = 0}^{{L}_{{R}^{- 1}}} {r}_{n} {S}_{n + m}$$

L_{R}=length of reference 1-D sequence

The offset resulting in maximum correlation provides a definition of the index of the landmark in the 2-D trace from second viewing. When the landmark is being tracked in a sequence of frames, the index found in a given image frame may be used as the reference landmark for conducting the search in the next frame.

It should be noted that alternatives to the cross correlation function may be used for matching, for examples, minimum absolute difference and normalized cross correlation.

It should also be noted that anatomical features other than blood vessel traces may be used in generating the 1-D function described above. In another embodiment, for example, the thickness of a blood vessel can be used to generate the 1-D function. Measurement of the vessel thickness could be done as part of the automatic segmentation and tracing of the blood vessel.

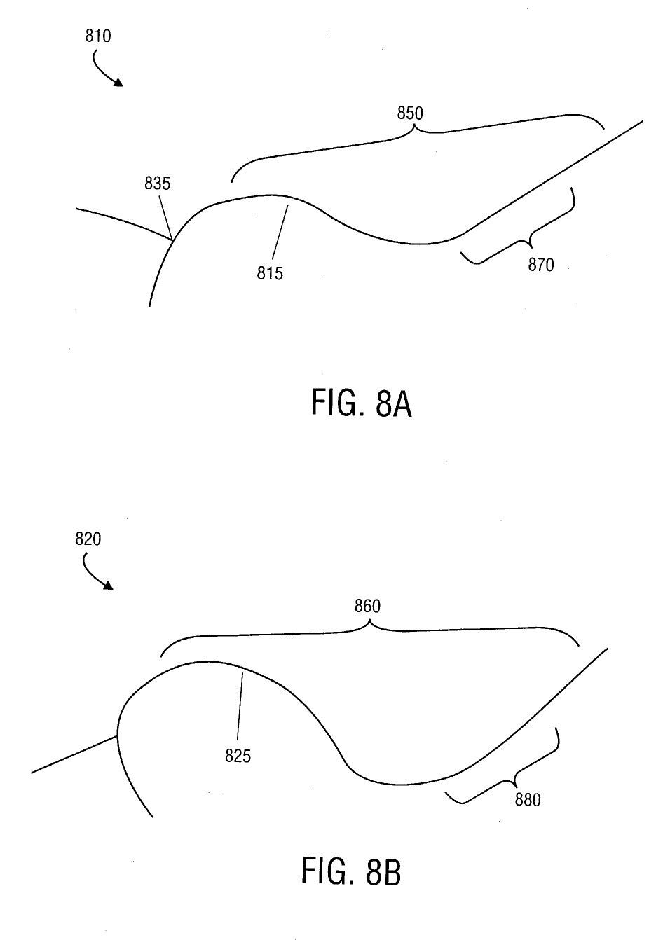

FIG. 8A illustrates one embodiment of a blood vessel having a first viewed position 810. In one embodiment, the first viewed position 810 is displayed on display unit 520 of FIG. 5. The blood vessel may be a coronary vessel, which may include an anatomical landmark containing a vascular intervention site. A first viewed position of an anatomical landmark 850 containing vascular treatment site 870 may be selected. Selecting the first viewed position of the anatomical landmark 850 may be accomplished by using virtual markers, such as the virtual markers 650 of FIG. 6A.

After a certain period of time, the actual position of the blood vessel within a patient's body may change. This may be a result of a heartbeat, for example, where the force or motion of the heartbeat re-positions the blood vessel. Thus, a second viewed position of the blood vessel 820 may be identified and displayed, as shown in FIG. 8B. Because the actual position of the blood vessel may have changed, the anatomical landmark may now have a second viewed position 860 relative to the first viewed position 850 in the body but still on the same segment of the blood vessel. In one embodiment, the actual position of the anatomical landmark may not have changed but the second viewed position 860 may appear different from the first viewed position 850 because of a change in imaging geometry, such as a change in X-ray angle or a change in magnification. In another embodiment, the actual position of the vascular intervention site has changed but the second viewed position 860 appears similar to the first viewed position 850 because of a change in imaging geometry.

The blood vessel may have an anatomical feature, such as a bend or curve in the vessel, whose actual position may change with a change in actual position of the blood vessel. The anatomical feature may have a first viewed position 815, as shown in FIG. 8A. After a change in the viewed position of the blood vessel, the anatomical feature may have a second viewed position 825, as shown in FIG. 8B. The actual position of the anatomical feature may not have changed, but the second viewed position 825 may appear different from the first viewed position 815 because of a change in imaging geometry, similar to the discussion above in relation to FIG. 8A. In another embodiment, the anatomical feature may be a branching point of the vessel, as illustrated by a first viewed position of a branching point 835 in FIG. 8A. The anatomical feature may be one of many other things, including a difference in vessel thickness relative to the vascular treatment site.

A pattern of the anatomical landmark including the vascular treatment site may be determined by use of mathematical calculations, as described above in relation to FIGS. 7A-7C. In one embodiment, a pattern of a first viewed position of an anatomical landmark including a first viewed position of a vascular intervention site 850 is determined.

After a certain period of time, a second viewed position of the blood vessel 820 may be displayed. A pattern of the second viewed position of the anatomical landmark 825 may be determined, as discussed above in relation to FIGS. 7A-7C. The patterns of the first and second viewed positions of the anatomical landmark may then be matched. This matching may yield an identification of a second viewed position of the anatomical landmark 860 containing vascular treatment site 880. The second viewed position of the anatomical landmark 860 may be identified on a display. This procedure may be executed repeatedly, for example, once every time a new image is displayed.

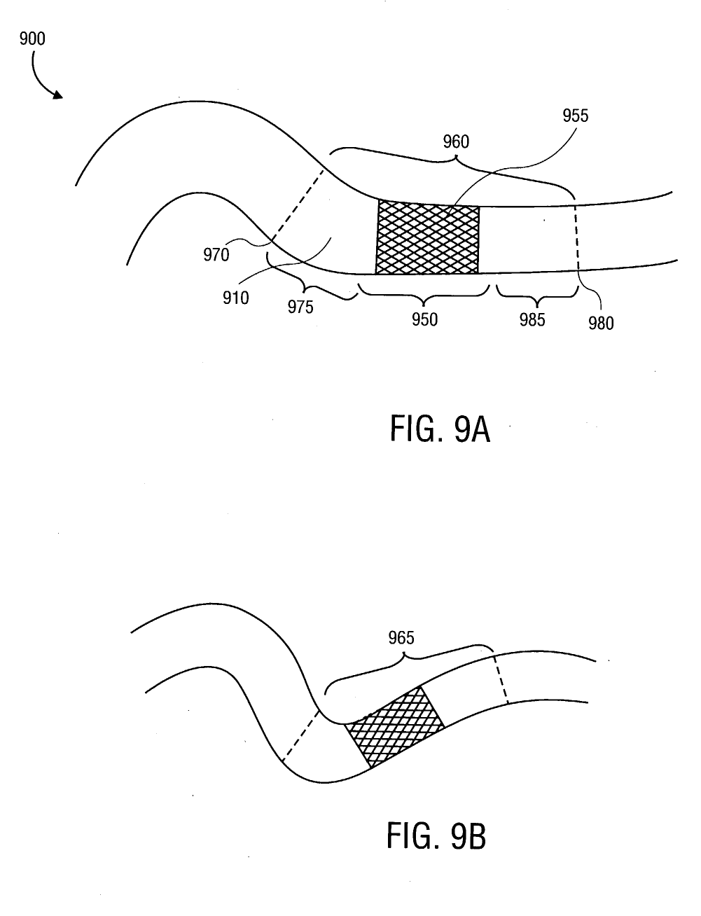

FIG. 9A illustrates one embodiment of a first viewed position of a blood vessel 900 having a first viewed position of a stent 955 located within a first viewed position of a vascular intervention site 950. Intervention site 950 is typically longer than the stent 955 that is used. A first viewed position of a proximal margin 975 may include the area between a first viewed position of a proximal edge 970 and the first viewed position of the vascular intervention site 950. A first viewed position of a distal margin 985 may include the area between a first viewed position of a distal edge 980 and the first viewed position of the vascular intervention site 950. A first viewed position of an anatomical landmark 960 may be defined by the first viewed position of the proximal edge 970 and the first viewed position of the distal edge 980. In one embodiment, anatomical landmark 960 corresponds to a desired vascular treatment site. The first viewed position of the anatomical landmark 960 may be larger than the first viewed position of the vascular intervention site 950. A user may desire the anatomical landmark, and thus the vascular treatment site, be larger than the vascular intervention site to account for one of several things that could happen to prevent complete treatment, such as uncertainty due to movement of radioactive seeds during radiotherapy.

The first viewed position of the anatomical landmark 960 may contain a first viewed position of an anatomical feature 910. Subsequent to selecting the first viewed position of the anatomical landmark 960, a first pattern of the first viewed position of the anatomical landmark 960 may be determined. At a later time, for example as part of a subsequent angiogram, a pattern of a second viewed position of the anatomical landmark 965 may be determined and matched with the first pattern. This matching may yield an identification of the vascular treatment site. The vascular treatment site may then be identified on a display, for example, through the use of virtual markers.

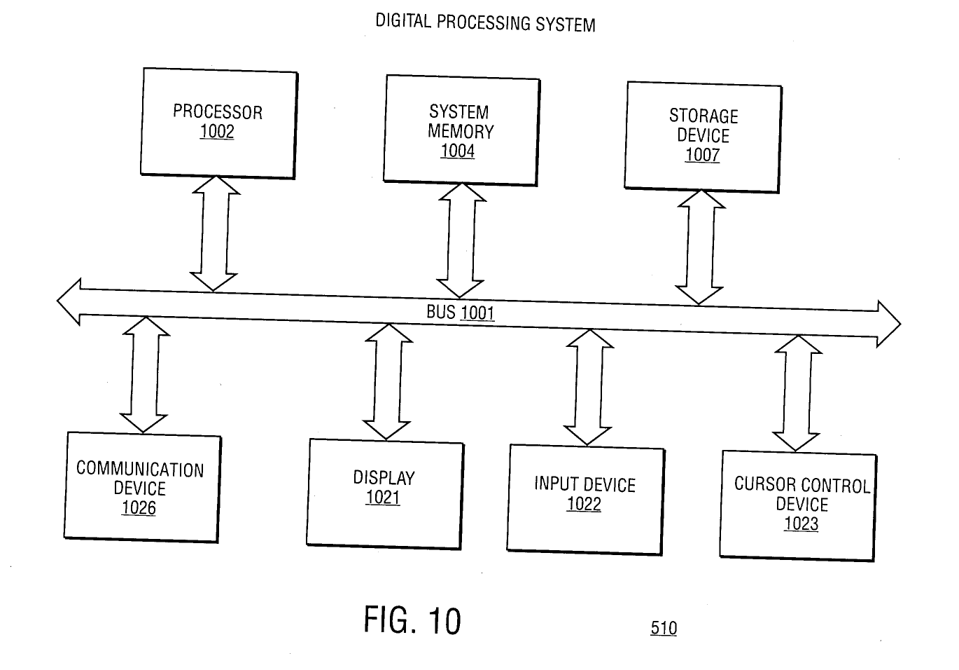

FIG. 10 illustrates one embodiment of digital processing system 510 of FIG. 5 representing an exemplary workstation, personal computer, laptop computer, handheld computer, personal digital assistant (PDA), closed-circuit monitoring box, etc., in which features of the present invention may be implemented.

Digital processing system 510 includes a bus or other means 1001 for transferring data among components of digital processing system 510. Digital processing system 510 also includes processing means such as processor 1002 coupled with bus 1001 for processing information. Processor 1002 may represent one or more general-purpose processors (e.g., a Motorola PowerPC processor and an Intel Pentium processor) or special purpose processor such as a digital signal processor (DSP) (e.g., a Texas Instruments DSP). Processor 1002 may be configured to execute the instructions for performing the operations and steps discussed herein. For example, processor 1002 may be configured to execute instructions to cause the processor to track vascular intervention sites.

Digital processing system 510 further includes system memory 1004 that may include a random access memory (RAM), or other dynamic storage device, coupled to bus 1001 for storing information and instructions to be executed by processor 1002. System memory 1004 also may be used for storing temporary variables or other intermediate information during execution of instructions by processor 1002. System memory 1004 may also include a read only memory (ROM) and/or other static storage device coupled to bus 1001 for storing static information and instructions for processor 1002.

A storage device 1007 represents one or more storage devices (e.g., a magnetic disk drive or optical disk drive) coupled to bus 1001 for storing information and instructions. Storage device 1007 may be used for storing instructions for performing the steps discussed herein.

In one embodiment, digital processing system 510 may also be coupled via bus 1001 to a display device 1021, such as a cathode ray tube (CRT) or liquid crystal display (LCD), for displaying information to the user. Such information may include, for example, graphical and/or textual depictions such as virtual markers on an angiogram display representing the edges of a vascular treatment site. An input device 1022, such as a light pen, may be coupled to bus 1001 for communicating information and/or command selections to processor 1002. Another type of user input device is cursor control 1023, such as a mouse, a trackball, or cursor direction keys for communicating direction information and command selections to processor 1002 and for controlling cursor movement on display 1021.

A communications device 1026 (e.g., a modem or a network interface card) may also be coupled to bus 1001. For example, the communications device 1026 may be an Ethernet card, token ring card, or other types of interfaces for providing a communication link to a network, such as a remote diagnostic or monitoring system, for which digital processing system 510 is establishing a connection.

It will be appreciated that the digital processing system 510 represents only one example of a system, which may have many different configurations and architectures, and which may be employed with the present invention. For example, some systems often have multiple buses, such as a peripheral bus, a dedicated cache bus, etc.

The method and apparatus discussed herein may enable users to more effectively treat patients with radiotherapy. Users may track the location and extent of a vascular treatment site, thus allowing for more complete and more effective radiotherapy to the treatment site. Full treatment to the treatment site is significant in reducing the possibility of restenosis and edge effect. The method and apparatus discussed herein are not limited to use only with radiotherapy and may be used with other types of therapies, for example, drug coated stent therapy.

In the foregoing specification, the invention has been described with reference to specific exemplary embodiments thereof. It will, however, be evident that various modifications and changes may be made thereto without departing from the broader spirit and scope of the invention as set forth in the appended claims. The specification and drawings are, accordingly, to be regarded in an illustrative sense rather than a restrictive sense.

1. A method, comprising:
selecting an anatomical landmark inside a patient;
marking the anatomical landmark on a first image of an angiogram display; and
identifying the anatomical landmark on a second image of the angiogram display.

2. The method of claim 1, wherein the anatomical landmark comprises a vascular treatment site corresponding to a vascular intervention site.

3. The method of claim 1, wherein identifying comprises locating and displaying the anatomical landmark.

4. The method of claim 3, wherein displaying comprises displaying at least one virtual marker representing the anatomical landmark.

5. The method of claim 2, wherein identifying comprises displaying a first virtual marker in alignment with a distal edge of the vascular treatment site and a second virtual marker in alignment with a proximal edge of the vascular treatment site.

6. The method of claim 4, wherein at least one virtual marker compensates for a change in imaging geometry.

7. The method of claim 1, wherein the first image and the second image are each displayed on a single angiogram display.

8. The method of claim 7, wherein the angiogram display is a split-screen display.

9. The method of claim 1, wherein the first image is displayed on a first angiogram display and the second image is displayed on a second angiogram display.

10. The method of claim 1, wherein the anatomical landmark comprises a portion of a coronary vessel.

11. The method of claim 10, wherein the portion of the coronary vessel is a curved segment.

12. The method of claim 2, wherein a stent is located within the vascular intervention site.

13. The method of claim 12, wherein the vascular treatment site is larger that the vascular intervention site.

14. The method of claim 12, wherein the vascular treatment site is to be treated with endovascular brachytherapy.

15. The method of claim 2, wherein marking comprises placing at least one virtual marker on the angiogram display.

16. The method of claim 15, wherein marking comprises placing a first virtual marker in alignment with a distal edge of the vascular treatment site and placing a second virtual marker in alignment with a proximal edge of the vascular treatment site.

17. The method of claim 1, wherein selecting comprises selecting the anatomical landmark by a user.

18. The method of claim 6, wherein the change in imaging geometry comprises a change in X-ray angle.

19. The method of claim 6, wherein the change in imaging geometry comprises a change in magnification.

20. The method of claim 4, wherein each virtual marker has a color specific to that marker.

21. The method of claim 20, wherein each virtual marker has a corresponding partner virtual marker, and wherein each virtual marker has the same color as its corresponding partner virtual marker.

22. The method of claim 3, wherein displaying comprises displaying at least one virtual marker that compensates for any change due to new treatment.

23. An apparatus, comprising:
means for selecting an anatomical site; and
means for determining a first pattern of the anatomical site.

24. The apparatus of claim 23, further comprising:
means for determining a second pattern of the anatomical site;
means for matching the first pattern and the second pattern; and
means for identifying the anatomical site based on the matching.

25. The apparatus of claim 24, further comprising means for storing data comprising at least one pattern.

26. The apparatus of claim 25, further comprising means for recalling stored data comprising at least one pattern.

27. A method, comprising:
selecting an anatomical site; and
determining a first pattern that includes the anatomical site.

28. The method of claim 27, further comprising:
determining a second pattern that includes the anatomical site;
matching the first pattern and the second pattern; and
identifying the anatomical site based on the matching.

29. The method of claim 28, wherein identifying the anatomical site comprises identifying the anatomical site on a display.

30. The method of claim 27, wherein selecting comprises marking an image of the anatomical site on a display by a user.

31. The method of claim 29, wherein identifying comprises automatically marking an image of the anatomical site on a display.

32. The method of claim 29, further comprising adjusting the anatomical site identified on the display by a user.

33. The method of claim 31, wherein automatically marking comprises referencing a center point of the anatomical site and adding at least one predetermined margin.

34. The method of claim 28, wherein the anatomical site comprises a portion of a coronary vessel.

35. The method of claim 34, wherein the portion of the coronary vessel is a curved segment.

36. The method of claim 28, wherein the anatomical site comprises a vascular treatment site corresponding to a vascular intervention site.

37. The method of claim 36, wherein a stent is located within the vascular intervention site.

38. The method of claim 36, wherein the vascular treatment site is larger that the vascular intervention site.

39. The method of claim 36, wherein the vascular treatment site is to be treated with endovascular brachytherapy.

40. The method of claim 28, further comprising storing data comprising at least one pattern.

41. The method of claim 40, further comprising recalling stored data comprising at least one pattern.

42. A method, comprising:
determining a first pattern of an anatomical landmark, the anatomical landmark comprising an anatomical site;
determining a second pattern of the anatomical landmark; and
matching the first pattern with the second pattern to determine a definition of the anatomical landmark.

43. The method of claim 42, wherein the anatomical landmark comprises a portion of a coronary vessel.

44. The method of claim 43, wherein the portion of the coronary vessel comprises a curved segment.

45. The method of claim 42, further comprising identifying the anatomical site on a display based on the definition.

46. The method of claim 42, wherein determining a first pattern comprises marking an image of the anatomical site on a display by a user.

47. The method of claim 45, wherein identifying the anatomical site on a display comprises automatically marking an image of the anatomical site on a display.

48. The method of claim 45, further comprising adjusting the anatomical site identified on the display by a user.

49. The method of claim 45, wherein the display is an angiogram display.

50. The method of claim 42, further comprising storing data comprising at least one pattern.

51. The method of claim 50, further comprising recalling stored data comprising at least one pattern.

52. A machine-readable medium having stored thereon instructions, which when executed by a processor, cause the processor to perform the following comprising:
determining a first pattern of an anatomical landmark, the anatomical landmark comprising an anatomical site;
determining a second pattern of the anatomical landmark; and
matching the first pattern with the second pattern to determine a definition of the anatomical landmark.

53. The machine-readable medium of claim 52, wherein determining comprises creating a trace designating the centerline of the anatomical landmark.

54. The machine-readable medium of claim 52, wherein matching comprises extracting a one-dimensional function from each pattern.

55. The machine-readable medium of claim 54, wherein matching further comprises performing cross-correlation matching of the functions.

56. The machine-readable medium of claim 54, wherein matching further comprises performing normalized cross-correlation matching of the functions.

57. The machine-readable medium of claim 54, wherein matching further comprises performing minimum absolute difference matching of the functions.

58. An apparatus, comprising:
a processor to match a first pattern of an anatomical landmark with a second pattern of the anatomical landmark to determine a definition of the anatomical landmark;
a display device coupled with the processor to display the anatomical landmark.

59. The apparatus of claim 58, further comprising an input device coupled with the processor.

60. The apparatus of claim 59, wherein the input device marks the anatomical landmark on the display device.

61. The apparatus of claim 59, wherein the input device is a light pen.

62. The apparatus of claim 58, further comprising a memory to store machine-readable media containing instructions executable by the processor to determine a definition of an anatomical landmark.

63. The apparatus of claim 58, further comprising a communication device coupled with the processor to receive data comprising at least one pattern of an anatomical landmark.

64. The apparatus of claim 58, further comprising a communication device coupled with the processor to transmit data comprising at least one pattern of an anatomical landmark.

65. The apparatus of claim 63, further comprising a remote diagnostic system coupled with the communication device.

66. The apparatus of claim 64, further comprising a remote monitoring system coupled with the communication device.
